(19) United States
(12) Patent Application Publication
(10) Pub. No.: US 2024/0315961 A1
(43) Pub. Date: Sep. 26, 2024

(54) SEMI-SOLID CHEWABLE COMPOSITIONS AND METHODS OF MAKING AND USING THEREOF

(71) Applicant: SEATTLE GUMMY COMPANY, Seattle, WA (US)

(72) Inventors: Feng Wan, Issaquah, WA (US); William Brenden CARLSON, Shoreline, WA (US)

(21) Appl. No.: 18/014,755

(22) PCT Filed: Dec. 1, 2021
(86) PCT No.: PCT/US21/61458

§ 371 Date: Jan. 6, 2023

Related U.S. Application Data
(60) Provisional application No. 63/119,658, filed on Dec. 1, 2020.
(60) Provisional application No. 63/119,661, filed on Dec. 1, 2020.
(60) Provisional application No. 63/119,657, filed on Dec. 1, 2020.
(60) Provisional application No. 63/119,660, filed on Dec. 1, 2020.

(51) Int. Cl. A61K 9/00; A61K 31/167; A61K 31/192; A61K 47/26; A61K 47/40

(52) U.S. Cl. CPC A61K 9/0056; A61K 31/167; A61K 31/192; A61K 47/26; A61K 47/40

(57) ABSTRACT

A semi-solid chewable composition, comprising an herbal composition and a semi-chewable base. The herbal composition comprises anti-inflammatory, anti-microbial, analgesic, antiseptic, or antipyrelic herbal composition.

### CROSS-REFERENCES TO RELATED APPLICATIONS

This application claims the benefit of priority from, and hereby incorporates by reference the entire disclosure, co-pending US Provisional Applications for (1) Patent Ser. No. 63/119,657, filed Dec. 1, 2020; (2) Patent Ser. No. 63/119,661, filed Dec. 1, 2020; (3) Patent Ser. No. 63/119,658, filed Dec. 1, 2020; and (4) and Patent Ser. No. 63/119,660, filed Dec. 1, 2020.

### TECHNICAL FIELD

This application relates to semi-solid edible or chewable gel compositions with one or more bioactive such as acetaminophen incorporated therein.

### BACKGROUND

Unless otherwise indicated herein, the materials described in this section are not prior art to the claims in this application and are not admitted being prior art by inclusion in this section.

Pharmaceuticals are available in a variety of dosage forms for treating the diseases. Dosages that are formulated to take orally including tablets, capsules, soft-gels, powders, chewable tablets, and liquid suspensions.

Tablets, capsules and soft gels are difficult for individuals who have difficulties swallowing pills. This problem is magnified when the medications need to be taken 2-4 times per day to provide the desired therapeutic effect. Moreover, the need for a source of water or other liquid to assist with swallowing solid dosage forms can complicate administration.

Powders are often difficult to administrator and chewable tablets can be hard to chew especially for seniors and young children. In addition, powders and chewable tablets often have an unpleasant after-taste.

Liquid suspensions or solutions are sometimes used as an alternative to solid oral dosage forms. However, the dosing with liquid dosage forms is not precise, which can lead to the administration of too little or too much medications. In addition, liquid dosage forms are messy and often have a bitter taste, which could impact person compliance.

Semi-solid chewable (gummy) composition could deliver medications and bioactive with an easier consumption profile. However, convention gummy formulation is often packed with sugar and high in glycemic index, making it unhealthy and potentially dangerous for diabetic patients.

### SUMMARY

The following summary is illustrative only and is not intended to be in any way limiting. In addition to the illustrative aspects, embodiments, and features described above, further aspects, embodiments, and features will become apparent by reference to the drawings and the following detailed description.

In one aspect, the application provides semi-solid chewable gel composition comprising acetaminophen. In one embodiment, the semi-solid chewable gel composition comprises acetaminophen, a complexing composition, a binding composition, optionally a polymer stabilizer, and a gelling composition.

In one embodiment, the composition comprises an active pharmaceutical ingredient (API) composition comprising acetaminophen, a complexing composition comprising an amide, a cyclodextrin, cluster dextrin, polyether, cyclic polyether or a combination thereof, a binding composition comprising a mono- or di-saccharide, a sugar alcohol, an oligosaccharide, or a combination thereof, and a gelling composition in a sufficient amount to provide a cohesive gelled product.

The API composition may further comprise ibuprofen, naproxen, aspirin, caffeine, codeine phosphate, codeine, hydrocodone bitartrate, pentazocine hydrochloride, dihydrocodeine bitartrate, oxycodone hydrochloride, butalbital, propoxyphene hydrochloride, propoxyphene napsylate, dexbrompheniramine maleate, pseudoephedrine sulfate, clemastine fumarate, pseudoepherine hydrochloride, tramadol hydrochloride, oxycodone terephthalate, their derivative or a combination thereof.

In one embodiment, the chewable composition has from 0.15% to 25% by weight of the API composition. In one embodiment, the chewable composition has about 0.1%, 0.3%, 1.5%, 2.5%, 3.75%, 5%, 7%, 8%, 10.1%, 15.6%, 20.5%, from 2.5% to 5%, from about 5% to 20% by weight of the API composition.

In one embodiment, the chewable composition comprises acetaminophen from about 2 mg to about 1000 mg per dose. In one embodiment, acetaminophen is about 720 mg, 650 mg, 500 mg, 325 mg, 160 mg, 120 mg, 80 mg, 48 mg, 10 mg, 5 mg, or 2 mg per dose.

In one embodiment, the chewable composition comprises 80 mg acetaminophen and 40 mg ibuprofen, 160 mg acetaminophen and 80 mg ibuprofen, 500 mg acetaminophen and 250 mg of ibuprofen, and 800 mg acetaminophen and 400 mg ibuprofen per dose.

The complexing composition is configured to complex at least partially with the API increasing the solubility, modulating the flavor profile of the API, or both. The complexing composition may be capable of complexing with the API through coordinating, chelating, complexing, hydrogen-bonding, dipole-dipole interaction, van-der waals interaction, or a combination thereof. In one embodiment, the API complex is capable of masking and reducing the bitterness, astringent, metallic, or foul taste of the API. In one embodiment, the API complex is capable of increasing API's solubility in aqueous matrix therefore facilitating the incorporation of the API into the aqueous gummy matrix.

In one embodiment, the complexing composition may be a cyclic glucose molecule (alpha-, beta-, gamma-cyclodextrin), cluster dextrin, maltodextrin, resistant starch, an oligosaccharide (such as inulin or soluble or non-soluble dietary fiber), a polysaccharide (such as a herbal polysaccharide), nucleic acid (DNA or RNA), an nucleotide molecule, an amino acid or its derivative thereof, a peptide, or an amid. In one embodiment, the complexing composition comprises cyclodextrin, a nucleotide, resistant starch, or a combination thereof.

In one embodiment, the complexing composition comprises protein, peptide, amide or polyamide, cluster dextrin, cyclodextrin, polydextrose, resistant starch, polyethylene glycol, polyunsaturated hydrocarbons, polyunsaturated fatty acids, mica, talc, zeolite, cellulose, plant particles, calcium carbonate, diatomaceous earth, chitosan, or a combination thereof. In one embodiment, the complexing composition comprises cyclodextrin, a nucleotide, resistant starch, an amide, a peptide, or a combination thereof.

In one embodiment, the complexing composition comprises an amide. Example amide includes without limitation such as N-acetyl glucosamine, n-acetyl galactosamine, 2-deoxy-2-aminoglucose N-acetyl, sialic acid N-acetyl, iminosugar N-acetyl, daunosamine N-acetyl, 2-deoxy-2-aminogalactose N-acetyl, chitin, pectin, and amino acids.

Plant particles may be derived from various parts of a plant such as flower, fruit, seed, grain, nut, nutshell, root, leaves, or stems. In one embodiment, the plant particles comprise berry powder, nutshell powder, rice bran powder, strawberry powder, orange pulp or peel powder, lemon pulp or peel powder, citrus fruit powder, apple powder, pineapple powder, baobab fruit powder, various berry powders including without limitation cherry powder, raspberry powder, blackberry powder, goji berry powder, cranberry powder or blueberry powder. DNA rich plant powder may be preferred such as strawberry, which is an octoploid. In one embodiment, the complexing composition comprises strawberry DNA.

In one embodiment, the complexing composition comprises cluster dextrin or cyclodextrin. In one embodiment, the cyclodextrin comprises alpha-dextrin, beta-cyclodextrin, gamma-cyclodextrin, or a combination thereof. In one embodiment, the cyclodextrin comprises essentially gamma-cyclodextrin.

In one embodiment, the complexing composition may further include porphyrin, lactam, zinc (II) ion, ferrous ion, ferric ion, calcium(II) ion, magnesium(II) ion,

In one embodiment, the composition comprises API and cyclodextrin at a molar ratio of from about 1:1 to about 1:100, from about 1:1 to about 1:20, or any ration in between. In one embodiment, the molar ratio of API and cyclodextrin is about 1:2, 1:5, 1:8, or 1:10.

The binding composition may include sugar, low glycemic sugar, or sugar alcohol, which leads to the chewable composition to be sugared, low glycemic (low sugar), or sugar free.

In one embodiment, the binding composition may include at least 2 binding agents selected from saccharides or sugar alcohols. Example saccharides include monosaccharide, di-saccharide, tri-saccharide, or a combination thereof.

In one embodiment, the binding composition comprises sucrose, glucose, fructose, or a combination thereof. Example sucrose may be cane sugar, beet sugar, coconut sugar, or a combination thereof. Example glucose may include glucose power or glucose syrup such as corn syrup, tapioca syrup, rice syrup, coconut syrup, or a combination thereof.

In one embodiment, the chewable composition is essentially free of sucrose, glucose, or fructose.

In one embodiment, the binding composition comprises a low GI (glycemic index) mono-saccharide, a low GI disaccharide, a low GI tri-saccharide, or a combination thereof. In one embodiment, the low GI monosaccharide comprises a stereoisomer of D-fructose, D-glucose, or D-galactose. In one embodiment, the low GI monosaccharide comprises L-fructose, L-glucose, L-galactose, Allulose, Sorbose, Tagatose, or a combination thereof. In one embodiment, the low GI disaccharide comprises an isomer of D-maltose (1,4-diglucose) or an isomer of D-sucrose (1,2-fructose glucose). In one embodiment, the low GI disaccharide comprises trehalose, isomaltulose (isomaltuose), or a combination thereof. In one embodiment, the low GI tri-saccharide comprises raffinose.

In one embodiment, the binding composition may include allulose by a weight of not less than 30%, 40%, 50%, 55%, 60% or 70%. In one embodiment, the binding composition may include allulose by a weight from about 50% to about 60%.

In one embodiment, the binding composition may include trehalose by a weight of not less than 15%, 20%, 22%, 22.5%, 25%, 30%, 40%, 50%, 55%, or 60%. In one embodiment, the binding composition may include trehalose by a weight from about 20% to about 30%.

In one embodiment, the binding composition may include isomaltulose by a weight of not less than 5%, 10%, 15%, 20%, 22%, 22.5%, 25%, 30%, 40%, 50%, 55%, or 60%. In one embodiment, the binding composition may include isomaltulose by a weight from about 10% to about 30%.

In one embodiment, the binding composition may include trehalose and isomaltulose. The a ratio may be from about 3:1 to about 1:3. In one embodiment, the binding composition may include trehalose and isomaltulose in a ratio of 3:1, 2:1, 1:1, 1:2, 2:1, or 3:1.

In one embodiment, the binding composition comprises trehalose, isomaltulose, and allulose. In one embodiment, the binding composition consists essentially of trehalose, isomaltulose and a third low GI sugar selected from L-fructose, L-glucose, L-galactose, allulose, Sorbose, Tagatose, or a combination thereof.

In one embodiment, the binding composition consists essentially of tagatose, allulose, or a combination thereof. In one embodiment, the binding composition consists essentially of tagatose, isomaltulose, or a combination thereof. In one embodiment, the binding composition consists essentially of isomaltulose, allulose, trehalose, or a combination thereof.

In one embodiment, the semi-solid chewable composition may be sugar free. In one embodiment, the binding composition consists essentially of sugar alcohols. In one embodiment, the binding composition comprises mannitol, sorbitol, xylitol, lactitol, isomalt, maltitol, hydrogenated starch hydrolysates (HSH), glycerol, erythritol, or a combination thereof.

In one embodiment, the binding composition consists essentially of mannitol, maltitol, isomalt, or a combination thereof. In one embodiment, the binding composition consists essentially of mannitol, sorbitol, or a combination thereof. Binding composition consists essentially of mannitol, sorbitol, erythritol or a combination thereof. In one embodiment, the binding composition consists essentially of mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof.

In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof.

In one embodiment, the binding composition consists essentially of mannitol, fructose, isomalt, or a combination thereof. In one embodiment, the binding composition consists essentially of mannitol, N-acetylglucosamine, isomalt, or a combination thereof. In one embodiment, the binding composition consists essentially of mannitol, maltitol, sucrose, or a combination thereof. In one embodiment, the binding composition consists essentially of mannose, maltitol, xylitol, or a combination thereof.

In one embodiment, the binding composition consists essentially of trehalose, xylitol, sorbitol or a combination thereof. In one embodiment, the binding composition consists essentially of mannitol, isomaltulose, xylitol, sorbitol or a combination thereof. In one embodiment, the binding composition consists essentially of maltitol, allulose, or a combination thereof. In one embodiment, the binding composition consists essentially of maltitol, allulose, resistant starch or a combination thereof. In one embodiment, the binding composition consists essentially of maltitol, sorbitol, allulose, or a combination thereof. In one embodiment, the binding composition consists essentially of maltitol, tagatose, or a combination thereof. binding composition consists essentially of allulose, tagatose, maltitol, isomalt, or a combination thereof. In one embodiment, the binding composition consists essentially of isomalt, allulose, or a combination thereof. In one embodiment, the binding composition consists essentially of isomalt, sorbitol, allulose, or a combination thereof. In one embodiment, the binding composition consists essentially of isomalt, allulose, resistant maltodextrin or a combination thereof.

In one embodiment, the binding composition may comprise allulose, maltitol, or a combination thereof. The ratio of allulose and maltitol may be from about 1:1 to about 2:1, about 10:7, or about 10:6. In one embodiment, the binding composition may comprise maltitol, sorbitol or a combination thereof. The ratio of maltitol and sorbitol may be from about 1:1 to about 5:3. In one embodiment, the binding composition may comprise allulose, sorbitol or a combination thereof. The ratio of allulose and sorbitol may be from about 10:8 to about 2:1. In one embodiment, the binding composition may comprise allulose, xylitol or a combination thereof. The ratio of allulose and xylitol may be from about 10:8 to 2:1. In one embodiment, the binding composition may comprise maltitol, xylitol or a combination thereof. The ratio of maltitol and xylitol may be from about 8:10 to 3:2. In one embodiment, the binding composition may comprise allulose, crythritol or a combination thereof. The ratio of allulose and erythritol may be from about 1:1 to 2:1

In one embodiment, the binding composition is substantially sugar-free. In one embodiment, the binding composition is substantially free of any sugar having a glycemic index of more than 50. In one embodiment, the binding composition is substantially free of sugar alcohols.

In one embodiment, the binding composition has a glycemic index of more than 70. In one embodiment, the binding composition has a glycemic index of 220, 200, 170, 160, 120, 100, 80, or 70. In one embodiment, the binding composition has a glycemic index of less than 50, 30, 20, 15, 10, 8 or 5. In one embodiment, the binding composition has a glycemic index of 0. In one embodiment, the chewable composition may have zero calorie.

In one embodiment, the chewable composition has a glycemic index of more than 70. In one embodiment, the chewable composition has a glycemic index of 220, 200, 170, 160, 120, 100, 80, or 70. In one embodiment, the chewable composition has a glycemic index of less than 50, 30, 20, 15, 10, 8, or 5. In one embodiment, the chewable composition, having a glycemic index of about 0.

In one embodiment, the chewable composition comprises from about 50% to 85% by weight of the binding composition. In one embodiment, the chewable composition comprises from about 60% to 80%, 65% to 75%, 65% to 60%, 67% to 71% or 68% to 69% w/w of the binding composition. In one embodiment, the chewable composition comprises from about 50%, 67%, 68%, 70%, 75%, 80% w/w of the binding composition.

For the chewable composition that is substantially free of glucose, sucrose, and fructose, the composition may further comprise a polymer stabilizer, wherein the polymer stabilizer is water-soluble. Through extensive research, the applicant discovered that the semi-solid chewable gel compositions comprising essentially sugar alcohol or low glycemic sugars as binding agents are prone to crystallization leading to insatiability of the formulation. The applicant further discovered that certain concentration of polymer stabilizers may be used to promote the thermodynamic stability of the formulation and reducing the crystallization.

Through significant and extensive experimentation, Applicant discovered that a stabilizing polymer significantly extends the stability of a low-sugar or sugar-free chewable composition formulations for about 1.5, 2, 3, 4, 5, 7, 8, 9, or over 10 times when compared to same formulations without the polymer stabilizer. For example, the stability may be extended from about 2 weeks to about 8months, about 3 months to about 9 months, about 6 months to about 12 months, about 5 months to about 14 months, about 9 months to over 24 months, about 10 months to over 36 months.

The water-soluble polymer stabilizer may include a polysaccharide, a polyvinyl alcohol, a polyalcohol, a vinyl alcohol, a peptide, a cationic polymer, a polyphenol, or a combination thereof.

In one embodiment, the polymer stabilizer comprises a polymer of monosaccharide monomers, wherein the monosaccharide monomer comprises glucose monomer or mannose monomer linked through glyosidic bonds substantially free of alpha-1,4-glycosidic bond.

In one embodiment, the polymer stabilizer may be a polymer of monosaccharide monomers (i.e. polysaccharide) selected from glucose, fructose, mannose, galactose, arabinose, rhamnose, xylose, galacturonate, glucuronate, N-acetylgalactosamine, N-acetylglucosamine, or a combination thereof, and wherein the polymer comprises from about 5 to about 500 monosaccharide monomers. The polysaccharide may be cationic, anionic, or nonionic. It could be homo-polysaccharide or hetero-polysaccharide. In one embodiment, the polysaccharide comprises from about 5 to about 50 monomers.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccharide, fructo-oligosaccharide, galactomannan oligomers, oligomers of ribose, xylose, arabinose, rhamnose, or a combination thereof.

In one embodiment, the polysaccharide comprises alpha-mannose monomers, beta-mannose monomers, beta-glucose monomers, or a combination thereof. In one embodiment, the polysaccharide comprises a polymer of glucose monomer or mannose monomer linked through glycosidic bonds. In one embodiment, the glycosidic bond is substantially free of 1,4-alpha-glycosidic bond. In one embodiment, the glycosidic bond comprises 1,2-alpha glycosidic bond, 1,3-alpha glycosidic bond, 1,2-beta glycosidic bond, 1,3-beta glycosidic bond, or a combination thereof.

In one embodiment, the polymer stabilizer comprises soluble fiber from tapioca, soluble corn fiber, soluble fiber from chicory root, soluble fiber from dandelion, maltodextrin, resistant maltodextrin, 6-20 β-1,4-linked glucopyranose units, 6-20 β-1,3-linked glucopyranose units, 6-20 β-1,2-linked glucopyranose units, 6-20 a-1,3-linked glucopyranose units, 6-20 a-1,2-linked glucopyranose units, or combination thereof. In one embodiment, the polymer stabilizer comprises maltodextrin.

In one embodiment, the vinyl alcohol comprises a hydroxy methyl acrylate.

In one embodiment, the peptide comprises a collagen, a cationic peptide, or a combination thereof.

In one embodiment, the chewable composition includes from about 1% to 10%, 1% to 5%, 2% to 6%, 0.5% to 15% w/w of the polymer stabilizer. In one embodiment, the chewable composition comprises at least 2% w/w of the polymer stabilizer. In one embodiment, the chewable composition comprises from about 3% to 10%, 5% to 8%, 6% to 7% w/w of the polymer stabilizer.

In one embodiment, the binding composition consists essentially of isomalt, allulose, or a combination thereof with resistant maltodextrin as the stabilizer. In one embodiment, the binding composition consists essentially of isomalt, maltitol, or a combination thereof with polydextrose as the stabilizer.

In one embodiment, the weight ratio of the binding composition and the polymer stabilizer is from about 3:1 to about 20:1, or any ratio in between, including 5:1, 6:1, 7:1, 8: 1, 10:1, 12, 1, 15:1, 18:1, or 19:1. In one embodiment, the weight ratio of the binding composition and the polymer stabilizer from about 10:1 to 15. In one embodiment, the weight ratio of the binding composition and the polymer stabilizer may be from about 5:1 to 20:1, 6:1 to 15:1, 5:1 to 10: 1, 10: 1 to 12:1, 12: 1 to 15:1, 10:1 to 20:1, or from 8:1 to 18:1.

In one embodiment, the gelling composition comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

In one embodiment, the gelling composition comprises pectin. In one embodiment, the pectin has a methoxyl content (i.e., esterification degree or DE) not less than about 15%, 20%, 40%, 50% or 65%. In one embodiment, the methoxyl content is from about 15% to 40%, 15% to 25%, 16% to 24%, 30% to 70%, 50% to 65%, 55% to 65%, 59% to 63%, or 60% to 80%.

In one embodiment, the pectin has an amide content not less than about 15%, 20%, 30%, or 40%. In one embodiment, the amid content is from about 12% to 40%, 15% to 35%, 15% to 25%, 20% to 25%, 25% to 40%.

In one embodiment, the total of the methoxyl content and the amide content is from about 36% to 70%. In one embodiment, the methoxyl content is more than about 25% and the amide content is not less than about 20. In one embodiment, the methoxyl content is from about 16% to 24% and the amide content is from about 20% to 25% . In one embodiment, the methoxyl content is from about 56% to 66% and the amide content is from about 0.1% to 0.5%.

In one embodiment, the gelling composition comprises gelatin. In one embodiment, the gelling composition comprises pectin, gelatin, collagen, or a combination thereof. In one embodiment, the gelling composition comprises pectin and collagen in a ratio from about 1:1 to about 1:3. In one embodiment, the gelling composition comprises pectin and collagen in a ratio about 1:2.

In one embodiment, the chewable gel composition includes from about 0.5% to 10%, 1.5% to 2.5%, 0.5% to 1.5% w/w of the gelling composition. In one embodiment, the chewable gel composition includes from about 0.5% to 1.5% w/w of the gelling composition, wherein the gelling composition comprises carrageenan. In one embodiment, the semi-solid chewable gel composition includes from about 5% to about 10% by weight of the gelling composition, wherein the gelling composition comprises gelatin.

In one embodiment, the chewable gel composition further includes an herbal composition, an antioxidant composition, a vitamin composition, a mineral composition, an amino acid composition, a probiotics composition, or a prebiotics composition.

In one embodiment, the herbal composition comprises an anti-inflammatory herb. In one embodiment, the herbal composition comprises an adaptagen. Example herbs include without limitation ginger, turmeric, curcumin, boswellia serrate resin (Frankincense), white willow bark, green tea, pycnogenol (maritime pine bark), resveratrol, uncaria tomentosa (cat's claw), any ginseng species such as American ginseng, red panax ginseng, or Siberia ginseng, ashwagandha, Schisandra, Ginkgo Biloba, Cayenne, capsaicin (chilli pepper), black pepper, rosemary, Devils claw (Harpagophytum procumbens), Feverfew, Arnica montana, Hypericum (St John's Wort,) chamomilla, bromelain, clove, cinnamon, thyme, Astragalus, extract, powder, isolate or distillate thereof.

In one embodiment, the antioxidant composition comprises Vitamin E, Vitamin C, beta-carotene, gallic acid, selenium, selenium yeast, phenolics, anthocyanins, flavonoids, polyphenols, whey, bioflavonoids, theobromine, anthracenes, carotenoids, lutein, zeaxanthin, ginko biloba, berry extract, resveratrol, saffron, Sangre de grado (dragon's blood), cocoa, or derivatives thereof.

In one embodiment, the vitamin composition comprises vitamin A, B, C, D, E, K or a combination thereof. In one embodiment, the vitamin composition comprises Vitamin B9 (or folic acid), Vitamin D, Vitamin B3 (Niacin or Niacinamide), Vitamin C, or a combination thereof.

In one embodiment, the mineral composition comprises salts of calcium, iron, zinc, magnesium, sodium, chloride, potassium, copper, molybdenum, manganese, phosphorus, iodine, nickel, boron, or selenium, or a combination thereof. In one embodiment, the mineral composition comprises zinc citrate, zinc gluconate, zinc sulfate, zinc acetate, boron citrate, or a combination thereof.

The amino acid may be natural or non-natural occurring. In one embodiment, the amino acid composition comprises histidine, a branched chain amino acid such as leucine, iso-leucine, and valine, L-5 hydroxytryptophan (5-HTP), an essential amino acid such as lysine, methionine, phenylalanine, threonine, tryptophan, L-theanine, beta-alanine, and L-arginine, citrulline, carnitine, or its derivative thereof. In one embodiment, the amino acid composition comprises isoleucine or its derivative thereof.

In one embodiment, the prebiotic composition comprises gum arabic, chicory root powder or extract, wheat bran powder or extract, acacia gum, guar gum, Artichoke fiber, oat fiber, soluble corn fiber, inulin, resistant maltodextrin, resistant starch, or a combination thereof. In one embodiment, the probiotic composition comprises Lactobacillus acidophilus, Lactobacillus rhamnosus GG, Saccharomyces boulardii, Bifidobacterium bifidum, Bacillus coagulans, or a combination thereof.

In one embodiment, the semi-solid chewable gel composition may further include an additive selected from sweeteners, food acids, flavoring agents, coloring agents, humectants, bulking agents, fatty acids, triglycerides, plasticizers, thickeners, preservatives, or and a mixture thereof.

In one embodiment, the sweetener comprises xylitol, artificial sweeteners, saccharin, saccharin salts, cyclamic acid, cyclamic acid salts, aspartame, sucralose, acesulfame, rebaudioside A, rebaudioside B, rebaudioside C, rebaudioside D, rebaudioside E, dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium and other salts, aspartame, alitame, saccharin, neohesperidin dihydrochalcone, cyclamate, neotame, N-[N-[3-(3-hydroxy-4-methoxyphenyl)propyl]-L-.alpha.-aspartyl]-L-phenylalanine 1-methyl ester, N-[N-[3-(3-hydroxy-4-methoxyphenyl)-3-methylbutyl]-L-alpha-aspartyl]-L-phenylalanine 1-methyl ester, N-[N-[3-(3-methoxy-4-hydroxyphenyl)propyl]-L-alpha-aspartyl]-L-phenylal-anine I-methyl ester, salts thereof, licorice or its extracts or isolates, or a mixture thereof.

In one embodiment, the chewable gel composition is substantially free of artificial sweeteners, non-sugar sweeteners or sugar substitutes.

In one embodiment, the chewable composition may further include a coating composition. In one embodiment, the coating composition comprises isomalt, allulose, tagatose, xylitol, erythritol, sorbitol, mannitol, or a combination thereof. In one embodiment, the coating composition may have a particle size from about 0.6 mm to about 0.75 mm. In one embodiment, the coating composition may have a particle size of about 400 microns.

In one embodiment, the chewable composition may include the binding composition comprising maltitol and at least one of allulose, xylitol, erythritol, maltitol, sorbitol, and mannitol, and the coating composition including maltitol.

The pH of the chewable composition may be from about 3 to 5, 2 to 6, 2 to 4, 3 to 4, 2 to 5, or any number in between. In one embodiment, the pH of the composition is less than 5. In one embodiment, the pH of the composition is more than 7. In one embodiment, the pH of the composition is about 9.

In one embodiment, the gelling composition comprises pectin. In one embodiment, the pH of the composition is less than 3, 3.5, or 4. In one embodiment, the pH of the composition is more than 3 or 3.5. In one embodiment, the pH of the composition is more than 4 or 4.5. In one embodiment, the pH of the composition is about 7.

In one embodiment, the gelling composition comprises gelatin. In one embodiment, the pH of the composition is more than 6.

In one embodiment, the chewable composition may include the API composition comprises acetaminophen or a derivative thereof, the complexing composition comprises N-acetyl glucosamine and cyclodextrin, wherein the acetaminophen and the complexing composition have molar ratio from about 1:1 to about 1:10. In one embodiment, the binding composition comprising maltitol, mannitol and isomalt and the polymer stabilizer comprises polydextrose soluble fiber, wherein the binding composition and the polymer stabilizer have a w/w ratio from about 20:1 to about 4:1, from about 10:1 to 5:1, from about 15:1 to about 10:1, from about 8:1 to about 4:1. In one embodiment, the binding composition may include maltitol and allulose.

In one embodiment, the chewable composition comprises from about 80 mg to about 560 mg acetaminophen. The binding composition comprises glucose, sucrose and optionally fructose.

In one embodiment, the chewable composition comprises from about 20 mg, 40 mg, 80 mg, 160 mg, 320 mg, 480 mg, or 560 mg acetaminophen with N-acetyl glucosamine and cyclodextrin as the complecing agent and with a polymer stabilizer. In one embodiment, the binding composition comprises sugar alcohol. In one embodiment, the binding composition comprises allulose, tagatose, a sugar alcohol or a combination thereof. The polymer stabilizer comprises polydextrose, soluble corn fiber, soluble tapioca fiber, inulin or beta-glycan.

In another aspect, the application provides methods of making the chewable composition. In one embodiment, the method includes the steps of dividing the binding composition into a first binding portion and a second binding portion, combining a first mixture and water and heating to at a first elevated temperature to provide a first solution, wherein the first mixture comprises the first binding portion and optionally the polymer stabilizer, combining the second mixture and water at a second elevated temperature to provide a second solution, wherein the second mixture comprises the gelling composition with the second binding composition that is equal to, twice, three time, or four times the mass of the gelling composition, and the complexing composition, mixing the second solution into the first solution at a third elevated temperature to provide a third mixture, wherein the third mixture has a Brix number from about 80 to about 85 or from about 78 to about 86, adjusting pH of the third solution with a buffer salt to from about 3 to about 7.

In one embodiment, the method may further include the step of adding coloring agent, flavoring agent, or a combination thereof into the third mixture to provide a molding mix having a Brix from about 78 to about 86.

The API composition and the complexing composition may be added to the second mixture, the third mixture, or the molding mixture. In one embodiment, the API composition and the complexing composition may be added to the third mixture together with the coloring agent or flavoring agent.

In one embodiment, the method may further include the step of adding the molding mix to a preformed shaped cavity. In one embodiment, the method may further include the step of cooling the molding mix (or mixture) in the preformed shaped cavity until the molding mixture forms into the chewable composition piece.

The first, second and third elevated temperature may be independently from about 175° F., to about 275° F., from 175° F. to about 200° F., from 170° F. to about 210° F., or any temperature in between.

In one embodiment, the method comprises the following steps. In a first container, a gelling composition is mixed with a portion of a binding composition. Optionally, a buffering salt may be added. The components are mixed until homogeneous to provide a first mixture (Mix 1). In one embodiment, the gelling composition comprises pectin. In one embodiment, the portion of the binding composition comprises sorbitol and isomalt, or allulose and maltitol. In one embodiment, the buffering salt may include sodium citrate, potassium citrate, or a combination thereof.

In a second container, the remaining portion of the binding compositions added. In one embodiment, the remaining portion of the binding composition comprises sorbitol, isomalt, and mannitol. The components are mixed until homogeneous to provide a second mixture (Mix 2).

In a third container, a food acid is dissolved in an aqueous solution with additives such as coloring agent and flavoring agent. In one embodiment, the food acid comprises citric acid, malic acid, acetic acid, or a combination thereof. In one embodiment, the aqueous solution comprises water, ethanol, glycerol, or any combination thereof. All components are mixed and warmed until a homogenous solution is achieved to provide a third mixture (Mix 3). In one embodiment, the components are warmed to 175° F.

To a first reaction container, the API composition and the complexing composition may be added. In one embodiment, the API composition comprises acetaminophen or a derivative thereof. In one embodiment, the complexing composition comprises N-acetyl glucosamine and beta-cyclodextrin. Water is then added to the first reaction container. In one embodiment, the water may be heated first before adding to the first reaction container. In one embodiment, the water is heated to at least 200° F.

Then the Mix 1 is added to the first reaction container with stirring to provide a first solution. The mixture is stirred until the gelling composition fully swells and disperses. In one embodiment, the first solution may be brought to a light boil.

To a second reaction container, Mix 2 is added, followed with addition of water. The components are mixed to provide a second solution. In one embodiment, the second solution may be brought to a boil. Then, the first solution is combined with the second solution with mixing. The mixture is heated to a Brix number of at least 82 Brix. Then, Mix 3 is added dropwise with stirring to provide a molding mix having a Brix number of at least 82 Bix.

The molding mix was then added to a mold to provide individual gummy pieces. The mold may be a silicon mold, a starch mold, or a sugar alcohol mold. In one embodiment, the sugar alcohol mold is made by compacting a sugar alcohol composition powder or particles in a container to create a compacted a sugar alcohol mass, and stamping the compacted mass with a desirable shape to create mold cavities in the compacted mass. In one embodiment, the sugar alcohol composition comprises maltitol, isolmalt, or a combination thereof.

The molding mix may be injected or deposited into the mold cavities to form gummy pieces. The gummy piece may be any shape and size. The formed chewable composition may be in square shape, gumdrop shape, hexagon shape, partial ball shape, animal shape, cartoon shape, or any desirable shape. The shape may be hexagon, square, half ball, gumdrop, heart, bear, or any other shapes.

The formed composition may be from about 1 g to about 10 g, from about 2 g to about 7 g, from about 3 g to about 5 g, or any number in between. In one embodiment, the gummy piece may have a weight from about 3 g, 4 g, 5 g, 6 g, 7 g, 7.5 g, to about 8 g.

The gummy piece may be further coated with a coating composition. The coating composition may prevent gummy piece to stick with each other. The coating composition may include isomalt, maltitol, or other low glycemic sugar or sugar alcohol. In one embodiment, the coating composition comprises isomalt. In one embodiment, the coating composition comprises maltitol.

Through extensive experimentation, the Applicant discovered that the water solubility property of the coating composition is critical in maintaining gummy formulation stability. In one embodiment, the coating composition may have a water solubility of at least 2000 g/L, 1750 g/L, 1500 g/L, 1000 g/L, 500 g/L of water at room temperature. In one embodiment, the water solubility of the coating composition is at least 1500g/L. In one embodiment, the coating composition comprises isomalt, allulose, maltitol, maltodextrin, inulin, starch, bran, xylitol, sorbitol, tagatose, erythritol, or a combination thereof.

The gummy shape may be geometric or in animal, or any object shape. Example shape includes hexagon, square, half ball, gumdrop, heart, bear, cube, square, half ball, bar, or round disk.

The mold may be a silicon mold, a starch mold, or a sugar alcohol mold. In one embodiment, the sugar alcohol mold is made by compacting sugar alcohol powder or particles in a container to create a compacted a sugar alcohol mass, and stamping the compacted sugar alcohol mass with a desirable shape to create mold cavities in the compacted sugar alcohol mass. The molding mix may be injected or deposited into the mold cavities to form gummy pieces. The sugar alcohol may be maltitol, isomalt or a combination thereof.

In a further aspect, the application provides methods for treating a condition in a subject using a semi-solid chewable gel composition comprising acetaminophen by administering to the subject an effective amount of the composition. The condition may include pain, aches, fever, cramps, cold, inflammation, or a combination thereof.

### BRIEF DESCRIPTION OF THE DRAWINGS

The foregoing and other features of this disclosure will become more fully apparent from the following description and appended claims, taken in conjunction with the accompanying drawings. Understanding that these drawings depict only several embodiments arranged in accordance with the disclosure and are, therefore, not to be considered limiting of its scope, the disclosure will be described with additional specificity and detail through use of the accompanying drawings, in which:

FIG. 1 shows the chemical structure of acetaminophen;

FIG. 2 shows the amide resonance mechanism;

FIG. 3 shows the association of acetaminophen with N-acetylglucosamine through the amide moiety, wherein the nitrogen with a partial positive charge associates with the oxygen with a partial negative charge and the association of the two amide groups results in a six-member ring; and

FIG. 4 shows the structure of N-acetylgalactosamine.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

This disclosure is generally drawn, inter alia, to compositions, methods, and processes related to semi-solid chewable composition.

As used herein, “semi-solid chewable gel composition,” “chewable composition,” “gummy” and “gummy composition” are used interchangeably.

As used herein, “allulose” and “psicose” are used interchangeably.

As used herein, “isomaltulose” and “palatinose” are used interchangeably.

As used herein, “mix” and “mixture” may be used interchangeably.

### Active Pharmaceutical Ingredients (APIs)

Gummy can be used as an efficient delivery mechanism for an API. Firstly, gummy composition is easy to consume especially for patient populations having swallowing difficulty including without limitation pediatric and geriatric population. Second, gummy, if formulated to taste good, increases patient's compliance in taking medication. Third, when being consumed, gummy composition dissolves and coats the mucosal membrane allowing for direct absorption of APIs through a trans-mucosal process, potentially bypassing the digestion track and reducing first-pass effect.

In one embodiment, the API composition comprises acetaminophen (or paracetamol). Acetaminophen is an active ingredient that is an analgesic and antipyretic compound for symptom relief such as headache, muscular aches, backache, minor pain of arthritis, the common cold, toothache, premenstrual and menstrual cramps, migraine, temporarily reduces fever, or a combination thereof. Acetaminophen acts to reduce inflammation that can lead manifestation of pain. Acetaminophen is also used for severe pain, such as cancer pain and pain after surgery.

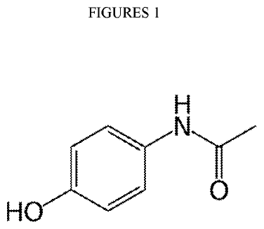

However, acetaminophen has an unpleasing taste. While a gummy delivery of acetaminophen would be advantageous due to rapid absorption and symptom relief, such product would not be popular if the API imparts the unpleasant taste to the gummy composition. FIG. 1 shows the chemical structure for acetaminophen. The amide moiety contributes to the bitterness as N is strongly alkaline, and alkaline functionality often activates the bitter taste receptors.

### Complexing Compositions

Complexing compositions may be useful to complex with active ingredients such as APIs or herbal extract or therefore masking or modulating flavor profile including reducing bitterness. The chewable composition includes a complexing composition that coordinates or complexes with acetaminophen molecule. In one embodiment, the complexing composition may include a molecule made of glucose units arranged in ring or crown structure. The acetaminophen molecule may partially fit inside the glucose ring. The complexing effect of the glucose ring structure greatly reduces the bitterness of the acetaminophen molecule in the gummy composition.

In one embodiment, the complexing composition comprises cyclic glucose including, for example, cluster dextrins and cyclic dextrin (i.e. cyclodextrin).

Cluster dextrins have a ring structure with many branches of long chains of glucose units pendent to the ring, forming a helical structure. The helical structure along with the ring structure is both able to chelate acetaminophen molecule. The chelation takes place by the phenyl groups on acetaminophen fitting inside the helical structure.

Cyclodextrin includes alpha, beta, and gamma-cyclodextrin. Alpha cyclodextrin rings form a crown. The inside of the crown is able to chelate hydrophobic structure such as the phenyl group of the acetaminophen molecule. The inside cavity of the cyclodextrin structure is largely hydrophobic which is favorable for complexing aromatic systems that are also hydrophobic. The formation of the chelate structure is endothermically favorable due to electrostatic interactions of the p system of the aromatic moiety with in the hydrophobic cavity and electronic interaction with the hydrogen atoms and glycidyl ether bonds. It is these electronic interactions between these systems of the cyclodextrin and the pi system that gives the favorable heat of formation.

The alpha, beta, and gamma cyclodextrins do not form the complex with acetaminophen equally. Acetaminophen forms stable complexes with all the cyclodextrins with gamma being the most stable complex and alpha being the least stable.

The coordination of acetaminophen by cluster dextrin molecules is more by chance. Cluster dextrin molecules have a broad range of cyclic ring and helical structures. Statistically a portion of cyclic and helical structures would chelate acetaminophen. The mechanism of chelation of acetaminophen by cluster dextrin structures is the same electronic interactions that occur as the alpha, beta, and gamma cyclodextrins.

In one embodiment, the complexing composition may include N-acetyl glucosamine, N-acetyl galactosamine, or a derivative or combination thereof. Through extensive research and experimentation, the applicant discovered that N-acetylglucosamine may coordinate at least partially with acetaminophen molecule in a gummy composition, which helps to reduce unpleasant flavors of acetaminophen. The resulting gummy composition is excellent in texture, taste, and flavor and allows for rapid delivery of the medication for fast relief of undesirable symptoms. In one embodiment, the ratio of acetaminophen and N-acetylglucosamine is about 2:1, 1:1, 1.5:1, 1.25:1, 0.75:1, to about 0.5:1.

N-Acetylglucosamine is an amino sugar where the hydroxy moiety on carbon 2 has been substituted with an amino moiety and subsequently amidated with acetic acid. Amides have the chemical structure

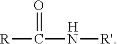

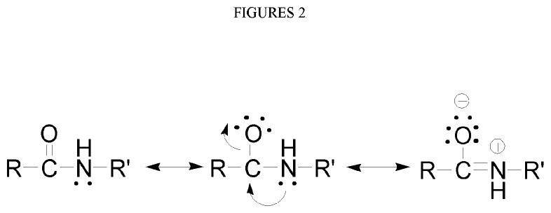

The chemical structure is such that there is an electron rich group, containing the nitrogen, adjacent to an electron deficient group, the carbonyl which contains the carbon and oxygen. Electron density can be transferred from the nitrogen to the carbonyl to form a resonance structure as shown in FIG. 2. The resonance structure is a Zwitterionic form of the amide structure where there is a positive charge on the nitrogen, a negative charge on the oxygen and a double bond between the nitrogen and carbon atoms.

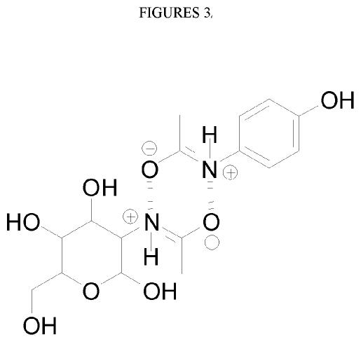

Acetaminophen contains a similar amide structure in N-acetylglucosamine. As shown in FIG. 3. Applicant discovered through extensive experimentation with the surprise finding that N-acetylglucosamine can associate with acetaminophen though the amide group, which reducing acetaminophen's unpleasant flavor.

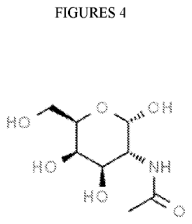

N-Acetylgalactosamine (GalNAc) is an amino sugar derivative of galactose having the following structure as shown in FIG. 4. Similar to N-acetylglucosamine, the association of N-acetyl group with acetaminophen through the amide moiety contributes to the reduction in acetaminophen medicinal taste.

### Binding Composition

Binding composition binds the semi-solid chewable gel (gummy) together through interaction with the gelling composition. The interaction may be through hydrogen bonding or through covalent bonding. In one embodiment, the binding composition comprises sugars, sugar derivatives, sugar alcohols, or a combination thereof. The binding composition may keep texture of the product soft by acting as a humectant.

The binding composition may be sugared, low-sugar of sugar free. In one embodiment, the binding composition may include at least 2 binding agents selected from a monosaccharide, a disaccharide, a tri-saccharide, or a sugar alcohol. In one embodiment, the binding composition includes at least 3 binding agents.

In one embodiment, the binding composition comprises sucrose, glucose, fructose, or a combination thereof.

In one embodiment, the binding composition comprises a mono-or di-saccharide (i.e., sugar) having a glycemic index of less than 80, 35, 30, 25, 20, 15, or 10, a sugar alcohol, or a combination thereof. In one embodiment, the semi-solid chewable composition is substantially free of sugar having a glycemic index of more than 35. In one embodiment, the semi-solid chewable composition has a glycemic index of not more than 8, 10, 15 or 20.

In one embodiment, the semi-solid chewable composition is substantially free of sucrose, fructose, glucose, sugar alcohol, sugar substitute, or non-sugar sweetener. Example sugar substitutes include, without limitation, sucralose, stevia extract or derivatives, monk fruit extract, licorice extract or derivative, tamarind extract or derivative, or their derivative thereof.

In one embodiment, the binding composition comprises a low GI sugar having a glycemic index (GI) of not more than 18, 20, 30, 35, or 80. In one embodiment, the low GI sugar comprises allulose, sorbose, tagatose, trehalose, isomaltulose, raffinose, or a combination thereof. In one embodiment, the binding composition comprising tagatose, allulose (also known as allulose), sorbose, isomaltulose (also known as isomaltulose), trehalose (also known as mycose), mannose, maltose, ribose, xylose, tetroses, pentoses, hexoses, heptoses, its acid form or a combination thereof. In one embodiment, the binding composition consists essentially of allulose, isomaltulose, and a third low GI sugar selected from a group consisting of trehalose, sorbose, tagatose, or a combination thereof. In one embodiment, the binding composition further comprises N-acetyl glucosamine.

Through extensive experimentation, Applicant developed processes formulation allulose, sorbose, tagatose, isomaltulose, trehalose in the gummy formulation allowing these sugars behave like conventional sugars but without the caloric significance and blood sugar impact of the convention sugars.

In one embodiment, the binding composition comprises allulose, trehalose and isomaltulose. In one embodiment, the binding composition comprises more than 20% isomaltulose. In one embodiment, the binding composition comprises from 15% to 35% isomaltulose. In one embodiment, the binding composition comprises not more than 75% of allulose. In one embodiment, the binding composition comprises from 45% to 60% allulose. In one embodiment, the binding composition comprises not more than 45% of trehalose.

In one embodiment, the binding composition comprises allulose and tagatose. In one embodiment, the binding composition comprises not more than 50% tagatose. In one embodiment, the binding composition comprises from 30% to 45% tagatose. In one embodiment, the binding composition comprises not more than 70% of allulose.

In one embodiment, the binding composition comprises isomaltulose and tagatose. In one embodiment, the binding composition comprises from 30% to 60% tagatose. In one embodiment, the binding composition comprises not more than 70% of isomaltulose.

Sugar alcohols are sweet, non-cariogenic, not digestible and provide fewer calories than many sugars. In some embodiments, sugar alcohols may mask other flavors. For example, mannitol may be used to mask bitterness. Mannitol masks bitterness by a mechanism that involves the endothermic nature of mannitol dissolving into water.

In one embodiment, the binding composition comprises essentially of sugar alcohols. Example sugar alcohols include sorbitol, mannitol, erythritol, xylitol, isomalt, maltitol, lactitol, and hydrogenated starch hydrolysates. In one embodiment, the binding composition comprises mannitol, maltitol, or isomalt. In one embodiment, the binding composition comprises mannitol, sorbitol, isomalt, or a combination thereof. In one embodiment, the binding composition consists essentially of mannitol, maltitol, sorbitol, or a combination thereof. In one embodiment, the binding composition consists essentially of mannitol, maltitol, xylitol, or a combination thereof. In one embodiment, the binding composition consists essentially of mannitol, xylitol, isomalt, or a combination thereof. In one embodiment, the binding composition consists essentially of mannitol, xylitol, sorbitol or a combination thereof. In one embodiment, the binding composition consists essentially of mannitol, sorbitol, isomalt, resistant starch or a combination thereof. In one embodiment, the binding composition consists essentially of mannitol, maltitol, sorbitol, maltodextrin or a combination thereof. In one embodiment, the binding composition consists essentially of mannitol, sorbitol, or a combination thereof. In one embodiment, the binding composition consists essentially of mannitol, sorbitol, erythritol or a combination thereof.

In one embodiment, the binding composition may include hydrogenated starch hydrolysates (HSH). HSH is a form of sugar alcohol from the hydrogenation of starch. The hydrogenation process yields a mixture of several sugar alcohols. HSH are nutritive sweeteners that provide 40-90% of the sweetness of sugar.

In one embodiment, the semi-solid chewable gel composition comprises from about 50% to about 90%, from about 60% to about 85% of the binding composition.

### Gelling Composition

In one embodiment, the gelling composition comprises pectin, gelatin, or a combination thereof. In one embodiment, gelatin may be combined with other hydrocolloids—pectin, agar, starch, gum Arabic—to create desired textures. In one embodiment, gelatin may be combined with gum arabic as the gelling composition.

In one embodiment, the gelling composition comprises starch such as amylose starch or modified starch. There are a variety of starch modification techniques, such as contacting starch with acid, sodium or potassium hydroxide, or oxidizing the starch.

In one embodiment, the gelling composition comprises agar. Agar may be combined with locust bean gum as a gelling composition Locust bean gum helps to prevent weeping of agar gels. The two polysaccharides from agar and locust bean gum synergistically interact with each other to form a strong gel that does not weep.

In one embodiment, the gelling composition comprises carageenans. Carrageenans or carrageenins are linear sulfated polysaccharides. Kappa-carrageenan has one sulphate group per disaccharide and forms strong, rigid gels in the presence of potassium ions. In one embodiment, locust bean gum may be used with kappa-carrageenan to prevent weeping. Gels formed from kappa-carrageenan and potassium ions are thermally reversible.

In one embodiment, the gelling composition comprises alginic acid or alginate. Alginate may form strong hydrogels when crosslinked with calcium ions.

### Polymer Stabilizer

The polymer stabilizer serves to stabilize the semi-solid chewable gel composition. In one embodiment, the polysaccharide may be water-soluble.

In one embodiment, the polymer stabilizer comprises a polysaccharide of mono-or di-saccharide monomers. In one embodiment, the monomers may include glucose, fructose, mannose, galactose, arabinose, rhamnose, xylose, galacturonate, glucuronate, N-acetylgalactosamine, N-acetylglucosamine, or a combination thereof. The polysaccharide comprises from about 5 to about 50 monosaccharide monomers.

In one embodiment, the polymer stabilizer comprises polydextrose, resistant starch, cellulose, maltodextrin, resistant maltodextrin, beta-glycan, soluble fiber, inulin, oligofructose, mannan-oligosaccharide, mannose oligosaccharide, galacto-oligosaccahride, fructo-ligosaccharide, galactomannan oligomers, oligomers of ribose, xylose, arabinose, rhamnose, hyaluronic acid, or a combination thereof.

Maltodextrin is an oligiosaccharide consisting of 6-20 glucose units. Maltodextrin can be in digestible or resistant forms. Digestible maltodextrin consists of a chain of 6-20 glucose units connected by alpha-1,4-glycosidic bonds. Resistant maltodextrin consists of a chain of 6-20 glucose units connected through alpha-1,2, alpha-1,3, beta-1,2, beta-1,3, and/or beta-1,4 glycosidic bonds. As the name implies, resistant maltodextrin resists digestion, as the enzyme amylase is inactive on the bonds that constitute resistant maltodextrin whereas amylase is active on the bonds that constitute maltodextrin. Maltodextrin provides for 4 Calories per gram whereas resistant maltodextrin provides for 1.6 Calories per gram. Through extensive experimentation, Applicant discovered that the inclusion of maltodextrin can help stabilize the gummy products against crystallization. In one embodiment, maltodextrin is about roughly 5% by weight

In one embodiment, the polymer stabilizer may include a polysaccharide derived from an herb. In one embodiment, the polysaccharide may derive from Cistanche deserticola, Astragalus membranaceus, Rubia cordifolia, Nerium indicum, Adhatoda vasica, Withania somnifera, and Glycyrrhiza glabra, aloe vera, Bletilla striata, Konjac, Goji berry, elderberry, or a combination thereof.

In one embodiment, the polymer stabilizer may include a mushroom polysaccharide. In one embodiment, the mushroom polysaccharide may derive from schizophyllum commune (Schizophyllum commue), Brazilian mushroom (Agarics blaze), Cordyceps sinensis (Cordyceps sinensis), glossy ganoderma (Ganoderma lucidum), rainbow conk (Coriolus versicolor), camphor tree sesame (Anthodia camphorate), Phellinus (Phellinus linteus), coral mushroom (Pleurotus citrinopileatus), mushroom (Lentinula edodes), Liu Songgu (Agrocybe aegerita), Hericium erinaceus (Hericium erinaceus), pleurotus eryngii (Pleurotus eryngiig), petal fine and soft (Sparrasis crispa), black fungus (Auricularia auricula), Asparagus (Flammulina velutipes) or a combination thereof. In one embodiment, the mushroom polysaccharide may include chitin, hemicellulose, α- and β-glucans, mannans, xylansand, or galactans. In one embodiment, the mushroom polysaccharides may include β-glucan polymers, with the main chain consisting of β-(1->3) linkages with some β-(1→6) branches as well as chitin, mannans, galactans, and xylans.

In one embodiment, the semi-solid composition may include from about 5% to about 15% of polymer stabilizer, or any number in between.

In one embodiment, the ratio of the binding composition and the polymer stabilizer is from about 8:1 to about 20:1, or any number in between.

### Coating Composition

The gummy pieces or semi-solid chewable composition disclosed herein may be coated with a coating composition. In one embodiment, the coating compositions may include sugar, sugar alcohol, or a combination thereof.

In one embodiment, the coating composition may include sucrose (sanding sugar).

For low sugar or sugar free composition, example sugars may include allulose, sorbose, tagatose, trehalose, and isomaltulose, or a combination thereof. Example sugar alcohols may include erythritol, sorbitol, mannitol, maltitol, isomalt, xylitol, or a combination thereof. In one embodiment, the coating composition comprises resistant starches, fibers, inulin, or a combination thereof. in one embodiment, the coating composition may include matitol, isomalte, allulose, or a combination herein.

The chewable composition may further comprise an herbal composition, an antioxidant composition, a vitamin composition, a mineral composition, an amino acid composition, a probiotics composition, or a prebiotics composition.

### Herbal Composition

Herbal composition may work with the API composition synergically enhancing API's therapeutic effect or reducing API's side effect. Herbal extract or derivatives may include flavanoids, allied phenolic compounds, polyphenolic compounds, terpenoids, alkaloids, sulphur-containing compounds, polysaccharides, flavone, flavonoids, quinone, or combinations thereof.

### Antioxidants

Antioxidants generally have anti-inflammatory activities. Antioxidant may include astaxanthin, carotenoids, coenzyme Q10 (“CoQ10”), flavonoids, glutathione, Goji (wolfberry), hesperidin, lacto-wolfberry, lignan, lutein, lycopene, polyphenols, selenium, vitamin A, vitamin C, vitamin E, zeaxanthin, or combinations thereof.

### Vitamins

Vitamins may include vitamin A, Vitamin B1 (thiamine), Vitamin B2 (riboflavin), Vitamin B3 (niacin, inositol hexanicotinate or niacinamide), Vitamin B5 (pantothenic acid or pantothenic acid salt), Vitamin B6 (pyridoxine, pyridoxal, or pyridovamine, or pyridoxine hydrochloride), Vitamin B7 (biotin), Vitamin B9 (folic acid), and Vitamin B12 (various cobalamins, commonly cyanocobalamin in vitamin supplements), vitamin C, vitamin D, vitamin E, vitamin K, K1 and K2 (i.e., MK-4, MK-7), folic acid, biotin, choline, or combinations thereof.

### Minerals

Minerals may include boron, calcium, chromium, copper, iodine, iron, magnesium, manganese, molybdenum, nickel, phosphorus, potassium, selenium, silicon, tin, vanadium, zinc, amino acid chelated minerals, yeast cell wall chelated minerals, or combinations thereof.

### Amino Acids

Amino acid may include for example alanine, arginine, asparagine, aspartate, cysteine, glutamate, glutamine, glycine, histidine, hydroxyproline, hydroxyserine, hydroxytyrosine, hydroxylysine, isoleucine, leucine, lysine, methionine, phenylalanine, proline, serine, taurine, threonine, tryptophan, tyrosine, valine, taurine, theanine, or combinations thereof. In one embodiment, taurine can be used to reduce bitterness by 50% when added at a concentration of 300 mM.

### Probiotics

Probiotic may include Aerococcus, Aspergillus, Bacteroides, Bifidobacterium, Candida, Clostridium, Debaromyces, Enterococcus, Fusobacterium, Lactobacillus, Lactococcus, Leuconostoc, Melissococcus, Micrococcus, Mucor, Oenococcus, Pediococcus, Penicillium, Peptostrepococcus, Pichia, Propionibacterium, Pseudocatenulatum, Rhizopus, Saccharomyces, Staphylococcus, Streptococcus, Torulopsis, Weissella, non-replicating microorganisms, or combinations thereof.

The composition may contain probiotic strains in an amount ranging from 10⁵ and 10¹² cfu/g. In one embodiment, the composition comprises between 10⁷-10¹⁰ cfu/g.

### Prebiotics

Useful prebiotics are lactose, inulin, fructo oligosacccharides, galacto oligosaccharides and xylo oligosaccharides. Prebiotics are neither to be hydrolysed nor absorbed by mammalian enzymes or tissues.

They selectively enrich beneficial bacteria in gut, can alter the intestinal micro-flora and its activities. Prebiotics can also change luminal or systemic aspects of the host defense system.

Prebiotics when combined with probiotics have many advantages. Basically, prebiotics selectively stimulate the growth of probiotics, which is dose and strain dependent. Prebiotics serve as a selective growth substrate for the probiotics strain during fermentation, during the period of storage, or during its passage through the gut.

Prebiotic may include for example acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, their hydrolysates, or combinations thereof.

### Humectants

A pharmaceutically acceptable humectant can include one or a mixture of humectants, such as, for example, glycerin, sorbitol and polyethylene glycol, for the gummy composition embodiments. The humectant content can be in the range of from 1% to 30% w/w and such as 2% to about 25% w/w.

Humectants are low molecular weight species that give the sensation of moisture in the chewable composition. The humectants mimic water in the chewable composition, which allows for very low water levels (2-6% ) in the chewable composition. The humectant can act as a moisturizing agent in the mouth. The humectant prevents the chewable composition from drying out and helps to maintain softness and shelf life. A very low vapor press is an important property of the humectant for the maintenance of softens so it does not evaporate out. Example humectants are glycerol (glycerin, 1,2,3-propantriol), propylene glycol (1,2-propandiol), aloe vera gel, glyceryl triacetate, and a-hydroxyacids (lactic acid).

### Sweeteners

Example sugar substitutes include saccharin, aspartame, sucralose and acesulfame K. In some embodiments, the sweetener used in the composition not only provides sweetness but also decreases the populations of bacteria in the mouth that lead to oral health problems. In one embodiment, the sweetener is selected from one or more of saccharin, aspartame, cyclamate, sucralose, Stevia, mannitol, sorbitol, xylitol and similar glycols.

### Plasticizers

Plasticizers impart flexibility by lowering the glass transition temperature of the polymers. They act in chewable composition decrease brittleness and increase chewiness. Some plasticizers that can be added to the chewable are lecithin, hydrogenated vegetable oils, glycerol mono, di and tri acetate ester, lanolin, methyl ester of the fatty acids, pentaerythritol mono, di, and tri acetate ester, rice bran wax, stearic acid, sodium and potassium stearates.

### Flavoring agents

An pharmaceutically acceptable flavoring agent can include one or a mixture of flavoring agents, such as for example bubble gum flavor, cherry flavor, grape flavor, anise oil, cassia oil, vanilla extract, vanilla creme, orange flavor, anethole, licorice, spearmint oil, phenylacetaldehyde diisobutyl acetal, and mixtures thereof, such as spearmint essential oil. Some flavoring agents can also act as sweeteners and can be use as such and include, for example, neohespiridin dehydrochalcone, xylitol, Sucralose, and mixtures thereof, such as xylitol. The flavoring agent can be in the range of from about of 0.05% w/w to about 3% w/w and such as about 0.5% w/w to about 1% w/w.

Flavors and colors are for sensory appeal. Flavor components in gum exist in liquid, powder or micro-encapsulated forms. Liquid flavor incorporations are either water-soluble, oil-soluble, or water-dispersible emulsions. The oil-soluble flavors remain in the gum longer, resulting in longer lasting flavor sensations, because the gum base is hydrophobic and attracted to oil-based components. Conversely, water soluble flavors dissolve into saliva and as such are extract out of the gum which leads to quicker loss in flavor.

In one embodiment, the flavoring agent is a phenolic flavoring agent selected from eucalyptol, thymol, methyl salicylate, menthol, chlorothymol, phenol, wintergreen oil, spearmint oil, peppermint oil and similar essential oils, and halogenated and other derivatives thereof.

### Flavoring Modulating Agents

Taurine, or 2-aminoethanesulfonic acid, is an organic compound that is widely distributed in animal tissues. It is a major constituent of bile and can be found in the large intestine, and accounts for up to 0.1% of total human body weight. Taurine can reduce bitterness by 50% when added at a concentration of 300 mM.

### Surfactants

A pharmaceutically acceptable surfactant can include one or a mixture of surfactants, such as, for example, certain nonionic, anionic and amphoteric surfactants and can include sulfate, sulfonate and phosphate ester surfactants (e.g., alkyl sulfonates having 10 to 18 carbon atoms and sulfates of monoglycerides of fatty acids having 10 to 18 carbon atoms) as well as salts and derivatives thereof including, for example, sodium lauryl sulfate (SLS) or sodium lauryl ether sulfate (SLES) as well as anionic taurate surfactants including, for example, sodium methyl cocoyl taurate and sodium methyl oleoyl taurate, PEG caster oils, and PEG hydrogenated caster oils, including, for example, PEG-60 Hydrogenated Castor Oil, Tweens including for example Tween 80, Tween 60, Tween 40. In one embodiment, the surfactant may be sodium lauryl sulfate (SLS) and sodium methyl cocoyl taurate, or a mixture of sodium lauryl sulfate (SLS) and sodium methyl cocoyl taurate. In one embodiment, the surface may be PEG-60 Hydrogenated Castor Oil. The surfactant content can be in the range of from about of 0.1% w/w to about 10% w/w and such as about 2% w/w to about 5% w/w.

In one embodiment, the surfactant is selected from one or more of sodium lauryl sulfate, sodium N-coco, N-methyl taurate, sodium N-lauroyl sarcosine, or a compatible dental detergent.

### Preservatives

A pharmaceutically acceptable preservative can include one or a mixture of preservatives, such as, for example, benzoic acid, sodium benzoate, methylparaben, propylparaben, sorbic acid and potassium sorbate. These preservative agents are generally present at levels ranging from about 0.01% w/w to about 2% w/w. The example preservative may be sodium benzoate.

### EXAMPLES

### Example 1. Acetaminophen Gelatin Gummy

9.9 g Acetaminophen, 0.5 g Sodium Benzoate, 0.5 g Potassium Sorbate, 240.3 g Sucrose, 283.1 g Glucose Syrup, 62.4 g Gelatin, 18.8 g Sorbitol, 2.0 g Kiwi Strawberry Flavor

The gelatin and sorbitol were shifted together until homogenous. The dry component mixture was added to 116.0 g of water rapidly with rapid stirring. The dry mix quickly absorbed the water and became a rubbery mass. The rubbery mass was added to a zip lock bag and heated at 160° F. until free of foam and a clear yellow.

Water was added to a container and heated to the boiling point. To the boiling water was added acetaminophen followed by potassium sorbate and sodium benzoate. The acetaminophen and other components quickly dissolved into the boiling water. The boiling acetaminophen solution was added glucose syrup with stirring. The solution was then brought to a boil. Sucrose was added to the aspirin/glucose solution. The solution was heated until 248° F. was reached. The solution was then cooled to 200° F. The gelatin solution was slowly added with stirring followed by the addition of the flavor. The mixture was stirred until homogenous. The solution was then added to silicone molds and the molds were placed in the refrigerator for 90 minutes. The result was chewable gummy product with 81 mg acetaminophen 7.5-gram gummy.

### Example 2. Acetaminophen Containing Pectin Gummy

5.500 g Sodium Bicarbonate, 3.673 g Potassium Hydroxide food grade, 33.33 g Acetaminophen, 145.3 g Citrus Pectin, 409.1 g Glucose Syrup, 310.3 g Sucrose, 15.0 g 50% citric acid, 4.5 g Pear, 0.6 g Green Food Color

Combine water, potassium hydroxide, sodium bicarbonate, and acetaminophen and mix thoroughly. Add the pectin. Stir until homogenous and let stand for 15 minutes. Bring to a boil for one minute. The glucose syrup was then added after the minute of boiling. The solution was brought back to a boil. The sucrose was then added and the system heated until Brix 87. The solution was cooled to 210° F. and a mixture of citric acid solution and pear flavor was added. These were mix into the batter until homogenous. Green food color was then added. The solution was then added to silicone molds and allowed to cool to room temperature. Excellent tasting gummies resulted. Each 7.5-gram gummy had roughly 250 mg acetaminophen per gummy.

### Example 3. Acetaminophen Containing Pectin Gummy

5.500 g Sodium Bicarbonate, 3.673 g Potassium Hydroxide food grade, 15.00 g Acetaminophen, 145.3 g Citrus Pectin, 409.1 g Glucose Syrup, 310.3 g Sucrose, 15.0 g 50% citric acid in water, 4.5 g Pear Flavor, 0.6 g Green Food Color

Combine water, potassium hydroxide, sodium bicarbonate, and acetaminophen and mix thoroughly. Mix pectin with three times mass sugar. Stir until homogenous. Bring water to a boil for one minute. Add the pectin mixture. The glucose syrup was heated until boiling and was then added after the minute of boiling. The solution was brought back to a boil. The remainder of sucrose was then added and the system heated until Brix 83. The solution was cooled to 210° F. and a mixture of citric acid solution and pear flavor was added. These were mix into the batter until homogenous. Green food color was then added. The solution was then added to silicone molds and allowed to cool to room temperature. Each 7.5-gram gummy had roughly 125 mg acetaminophen per gummy.

### Example 4. 80 mg Acetaminophen Containing Pectin Gummy

Pectin 22 g, Sucrose 332 1 g, Sodium Citrate 1.5 g, gamma-cyclodextrin 200 g, Acetaminophen 30 g, N-acetylglucosamine 20 g,

Water is added to a container and heated to 200° F. To the hot water are added the cyclodextrin and acetaminophen components. To a separate container are added pectin, fructose, mannitol and sodium citrate. These components are blended together until homogeneous. With stirring, the mixture of pectin and sugars is added to the heated water and allowed to dissolve. It takes about 5 minutes for the pectin to completely swell. Once the pectin is swelled the water is brought to a boil. In a separate container is added glucose syrup. The glucose syrup is brought to a boil and sucrose is added. The solution is brought to a boil and citric acid is added followed by the pectin solution. The solution is heated until Brix 83 has been reached at which point the color and flavors are added. Each 3.2-gram gummy had 80 mg of acetaminophen hydrochloride.

### Example 5. 160 mg Acetaminophen Containing Pectin Gummy

Pectin 22 g, Sucrose 332.1 g, Sodium Citrate 1.5 g, gamma-cyclodextrin 200 g, Acetaminophen 61.5 g, N-acetylglucosamine 20 g, Fructose 65 g, Mannitol 30 g, Glucose Syrup 333.33 g, Glycerol 5 g, Citric Acid 7 g, beta-Carotene Orange 2 g, Orange Flavor 4 g.

Water is added to a container and heated to 200° F. To the hot water are added the cyclodextrin and acetaminophen components. To a separate container are added pectin, fructose, mannitol and sodium citrate. These components are blended together until homogeneous. With stirring, the mixture of pectin and sugars is added to the heated water and allowed to dissolve. It takes about 5 minutes for the pectin to completely swell. Once the pectin is swelled the water is brought to a boil. In a separate container is added glucose syrup. The glucose syrup is brought to a boil and sucrose is added. The solution is brought to a boil and citric acid is added followed by the pectin solution. The solution is heated until Brix 83 has been reached at which point the color and flavors are added. Each 3.2-gram gummy had 160 mg of acetaminophen hydrochloride.

### Example 6. 80 mg Acetaminophen Containing Sugar Free Gummy

Pectin 22 g, Isomalt 332.1 g, Sodium Citrate 1.5 g, gamma-cyclodextrin 200 g, Acetaminophen 30 g, N-acetylglucosamine 20 g, Maltodextrin 65 g, Mannitol 30 g, Maltitol 333.33 g, Glycerol 5 g, Citric Acid 7 g, Red Color 2 g.

Crystalline maltitol, isomalt, maltodextrin, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the watermelon color and flavors are added. Each 3.2-gram gummy had 80 mg of acetaminophen.

### Example 7. 160 mg Acetaminophen Containing Sugar Free Gummy

Pectin 22 g, Isomalt 332.1 g, Sodium Citrate 1.5 g, gamma-cyclodextrin 200 g, Acetaminophen 65.5 g, N-acetylglucosamine 20 g, Maltodextrin 65 g, Mannitol 30 g, Maltitol 333.33 g, Glycerol 5 g, Citric Acid 7 g, beta-Carotene Orange 2 g, Orange Flavor 4 g.

Crystalline maltitol, isomalt, maltodextrin, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and 250 g water to create a syrup. The syrup is brought to a boil and citric acid is added followed by the pectin solution. The solution is heated until Brix 83 has been reached at which point the orange color and flavors are added. Each 3.2-gram gummy had 160 mg of acetaminophen.

### Example 8. 80 mg Acetaminophen Containing Pectin Gummy

Pectin 22 g, Sucrose 327.1 g, Sodium Citrate 1.5 g, gamma-cyclodextrin 200 g, Acetaminophen 30 g, Chitin Powder 30 g, Fructose 65 g, Mannitol 30 g, Glucose Syrup 328.33 g, Glycerol 5 g, Citric Acid 7 g, Red Color 2 g, Watermelon Flavor 4 g.

Water is added to a container and heated to 200° F. To the hot water are added the cyclodextrin and acetaminophen components. To a separate container are added pectin, fructose, mannitol and sodium citrate. These components are blended together until homogeneous. With stirring, the mixture of pectin and sugars is added to the heated water and allowed to dissolve. It takes about 5 minutes for the pectin to completely swell. Once the pectin is swelled the water is brought to a boil. In a separate container is added glucose syrup. Sucrose is blended with chitin powder. The glucose syrup is brought to a boil and the sucrose mixture is added. The solution is brought to a boil and citric acid is added followed by the pectin solution. The solution is heated until Brix 83 has been reached at which point the color and flavors are added. Each 3.2-gram gummy had 80 mg of acetaminophen hydrochloride.

### Example 9. 160 mg Acetaminophen Containing Pectin Gummy

Pectin 22 g, Sucrose 322.1 g, Sodium Citrate 1.5 g, gamma-cyclodextrin 200 g, Acetaminophen 61.5 g, Chitin Powder 40 g, Fructose 65 g, Mannitol 30 g, Glucose Syrup 313.33 g, Glycerol 5 g, Citric Acid 7 g, beta-Carotene Orange 2 g, Orange Flavor 4 g

Water is added to a container and heated to 200° F. To the hot water are added the cyclodextrin and acetaminophen components. To a separate container are added pectin, fructose, mannitol and sodium citrate. These components are blended together until homogeneous. With stirring, the mixture of pectin and sugars is added to the heated water and allowed to dissolve. It takes about 5 minutes for the pectin to completely swell. Once the pectin is swelled the water is brought to a boil. In a separate container is added glucose syrup. Sucrose is blended with chitin powder. The glucose syrup is brought to a boil and the sucrose mixture is added. The solution is brought to a boil and citric acid is added followed by the pectin solution. The solution is heated until Brix 83 has been reached at which point the color and flavors are added. Each 3.2-gram gummy had 80 mg of acetaminophen hydrochloride.

### Example 10. 80 mg Acetaminophen Containing Sugar Free Gummy

Pectin 22 g, Isomalt 327.1 g, Sodium Citrate 1.5 g, gamma-cyclodextrin 200 g, Acetaminophen 30 g, Chitin Powder 30 g, Maltodextrin 65 g, Mannitol 30 g, Maltitol 328.33 g, Glycerol 5 g, Citric Acid 7 g, Red Color 2 g, Watermelon flavor 4 g

Crystalline maltitol, isomalt, maltodextrin, chitin powder and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the watermelon color and flavors are added. Each 3.2-gram gummy had 80 mg of acetaminophen.

### Example 11. 160 mg Acetaminophen Containing Sugar Free Gummy

Pectin 22 g, Isomalt 322.1 g, Sodium Citrate 1.5 g, gamma-cyclodextrin 200 g, Acetaminophen 65.5 g, Chitin Powder 45 g, Maltodextrin 65 g, Mannitol 30 g, Maltitol 328.33 g, Glycerol 5 g, Citric Acid 7 g, beta-Carotene Orange 2 g, Orange Flavor 4 g

Crystalline maltitol, isomalt, maltodextrin, chitin powder, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the orange color and flavors are added. Each 3.2-gram gummy had 160 mg of acetaminophen.

### Example 12. 80 mg Acetaminophen Containing Sugar Free Gummy

Pectin 22 g, Isomalt 312.1 g, Sodium Citrate 1.5 g, gamma-cyclodextrin 200 g, Acetaminophen 30 g, N-acetylglucosamine 20 g, Maltodextrin 100 g, Mannitol 30 g, Maltitol 318.33 g, Glycerol 5 g, Citric Acid 7 g, Red Color 2 g.

Crystalline maltitol, isomalt, maltodextrin, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the watermelon color and flavors are added. Each 3.2-gram gummy had 80 mg of acetaminophen.

### Example 13. 80 mg Acetaminophen Containing Sugar Free Gummy

Pectin 22 g, Isomalt 287.1 g, Sodium Citrate 1.5 g, gamma-cyclodextrin 200 g, Acetaminophen 30 g, N-acetylglucosamine 20 g, Inulin 150 g, Mannitol 30 g, Maltitol 293.33 g, Glycerol 5 g, Citric Acid 7 g, Red Color 2 g.

Crystalline maltitol, isomalt, maltodextrin, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the watermelon color and flavors are added. Each 3.2-gram gummy had 80 mg of acetaminophen.

### Example 14. 80 mg Acetaminophen Containing Sugar Free Gummy

Pectin 22 g, Isomalt 262.1 g, Sodium Citrate 1.5 g, gamma-cyclodextrin 200 g, Acetaminophen 30 g, N-acetylglucosamine 20 g, Inulin 200 g, Mannitol 30 g, Maltitol 268.33 g, Glycerol 5 g, Citric Acid 7 g, Red Color 2 g.

Crystalline maltitol, isomalt, maltodextrin, and mannitol are mixed until homogenous. This is the sugar alcohol mixture. Water is added to a container and heated to 200° F. To the hot water are added the cyclodextrin and acetaminophen components. To a separate container are added pectin, 80 g of the sugar alcohol mix and sodium citrate. These components are blended together until homogeneous. With stirring, the mixture of pectin and sugar alcohols is added to the heated water and allowed to dissolve. It takes about 5 minutes for the pectin to completely swell. Once the pectin is swelled the water is brought to a boil. In a separate container is added the rest of the sugar alcohols and water to create a syrup. The syrup is brought to a boil and citric acid is added followed by the pectin solution. The solution is heated until Brix 83 has been reached at which point the watermelon color and flavors are added. Each 3.2-gram gummy had 80 mg of acetaminophen.

What is claimed is:

1. A semi-solid chewable gel composition, comprising,
an active pharmaceutical ingredient (API) composition, comprising acetaminophen,
a complexing composition, comprising an amide, a cyclodextrin, cluster dextrin, polyether, cyclic polyether, or a combination thereof, wherein the amide comprises N-acetyl glucosamine, N-acetyl galactosamine, a polyamide, lactam, chitin, amidated pectin, and ethylene-diamine-tetra acetamide, or a combination thereof,
a binding composition, comprising a mono- or di-saccharide, a sugar alcohol, an oligosaccharide, or a combination thereof, and
a gelling component in a sufficient amount to provide a cohesive gelled product.

2. The semi-solid chewable gel composition of claim 1, wherein the API composition further comprises ibuprofen, naproxen, or a combination thereof.

3. The semi-solid chewable composition of claim 1, comprising from 0.01% to 7.0% by weight of acetaminophen.

4. (canceled)

5. The semi-solid chewable gel composition of claim 1, wherein the complexing composition further comprises porphyrin, lactam, zinc(II) ion, ferrous ion, ferric ion, calcium(II) ion, magnesium(II) ion, or a combination thereof.

6. The semi-solid chewable gel composition of claim 1, wherein the complexing composition consists essentially of cyclodextrin, N-glucosamine, N-acetyl galactosamine, or a combination thereof.

7. (canceled)

8. The semi-solid chewable gel composition of claim 1, comprising from 0.1% to 25.0% by weight of the complexing composition.

9. The semi-solid chewable gel composition of claim 1, wherein the molar ratio of the API composition and the complexing composition is from 1:1 to about 1:5.

10. The semi-solid chewable gel composition of claim 1, wherein the binding composition comprises glucose, sucrose, fructose, sugar alcohol, allulose, isomaltose, trehalose, tagatose, raffinose, or a combination thereof.

11. The chewable composition of claim 1, further comprising a polymer stabilizer, and wherein the polymer stabilizer comprises a polymer of glucose monomer or mannose monomer linked through glycosidic bonds, wherein the glycosidic bond is substantially free of 1,4-alpha-glycosidic bond, and wherein the binding composition is essentially free of glucose, sucrose, or fructose, wherein the chewable composition.

12. (canceled)

13. The chewable composition of claim 11, wherein the binding composition consists essentially of allulose, isomaltose, trehalose, tagatose, raffinose, or a combination thereof.

14. The semi-solid chewable gel composition of claim 1, wherein the gelling component comprises gelatin, starch, pectin, gellan gum, guar gum, tapioca, protein, alginin, gum Arabic, carrageenan, guar, agar, agar-agar, carboxymethylcellulose, hydroxyethylcellulose, sago, alginate, locust bean gum, xanthan gum, or derivatives thereof.

15. The chewable composition of claim 1, wherein the gelling composition comprises pectin having a methoxyl content of less than 15% by weight of pectin or an amide content of more than 20% by weight of pectin.

16. (canceled)

17. The semi-solid chewable gel composition of claim 1, further comprising an herb composition, an antioxidant composition, a vitamin composition, a mineral composition, an amino acid composition, a probiotics composition, or a prebiotics composition.

18. The semi-solid chewable gel composition of claim 1, further comprising an additive selected from sweeteners, food acids, flavoring agents, coloring agents, humectants, bulking agents, fatty acids, triglycerides, plasticizers, thickeners, preservatives, or and a mixture thereof.

19. The semi-solid chewable gel composition of claim 1, wherein the gelling component comprises pectin wherein the pH of the composition is more than 4.

20. A method of making the semi-solid chewable gel composition of claim 1, comprising,
dividing the binding composition into a first binding portion and a second binding portion,
combining a first mixture and water and heating to at a first elevated temperature to provide a first solution, wherein the first mixture comprises the first binding portion,
combining the second mixture and water at a second elevated temperature to provide a second solution, wherein the second mixture comprises the gelling composition, the second binding portion, and the complexing composition, wherein the second binding portion is at least twice the mass of the gelling composition,
mixing the second solution into the first solution at a third elevated temperature to provide a third mixture, wherein the third mixture has a Brix number from about 78 to about 86, and
adjusting pH of the third solution with a buffer salt to from about 3 to about 7.

21. The method of claim 20, wherein the first mixture further comprises a polymer stabilizer.

22. The method of claim 20, wherein the second mixture or the third mixture further comprises the API composition, the complexing composition or both.

23. The method of claim 20, further comprising the step of adding a coloring agent, a flavoring agent, or a combination thereof into the third mixture to provide a molding mix having a Brix from about 78 to about 86.

24. A method of treating pains, fever, inflammation or migraine in a subject, comprising administering an effective amount of the semi-solid chewable gel composition of claim 1 to the subject.
